(19) United States
(12) Patent Application Publication
(10) Pub. No.: US 2025/0154089 A1
(43) Pub. Date: May 15, 2025

(54) METHODS FOR PREPARING OXYRESVERATROL AND DERIVATIVES THEREOF

(71) Applicant: Caliway Biopharmaceuticals Co., Ltd., New Taipei City (TW)

(72) Inventor: Yu-Fang LING, New Taipei City (TW)

(21) Appl. No.: 18/941,291

(22) Filed: Nov. 8, 2024

Related U.S. Application Data
(60) Provisional application No. 63/597,698, filed on Nov. 10, 2023.

(51) Int. Cl. C07C 37/20; C07C 37/62; C07C 39/21; C07F 9/50

(52) U.S. Cl. CPC C07C 37/20; C07C 37/62; C07C 39/21; C07F 9/505

(57) ABSTRACT

The present disclosure is directed to novel processes for producing resveratrol-type compounds (e.g., oxyresveratrol), salts thereof, hydrates thereof, and physical compositions thereof sans protecting group chemistry.

### CROSS REFERENCE TO RELATED APPLICATIONS

This application claims the benefit of the filing dates of U.S. Provisional Application No. 63/597,698, filed Nov. 10, 2023, the entire contents of which are incorporated by reference herein.

### BACKGROUND

Resveratrol and derivatives thereof, including oxyresveratrol, have been associated with anti-cancer and anti-aging properties. In particular, resveratrol-type compounds have been shown to have inhibitory tyrosinase activity, melanogenesis, antioxidant, and anti-inflammatory activities. Currently, resveratrol and derivatives thereof can be obtained naturally from biological sources or synthesized. Isolating the compounds from biological sources requires at least extraction from plant materials, and chromatographic separations. Conventional methods for preparing resveratrol-type compounds synthetically require protection/deprotection chemistry involving multiple synthetic steps, the use of metal catalysis, and combinations thereof. The synthesis of resveratrol-type compounds hinges on the formation of a central alkene. Classical synthetic methods for forming alkenes include coupling chemistry using metal catalysis (i.e., Suzuki coupling, Heck coupling, and McMurray reactions) requiring protecting group chemistry of phenolic hydroxyl groups of the reacting species. Therefore, a need remains for improved methods for obtaining resveratrol-type compounds.

### SUMMARY

The present disclosure is based, at least in part, on the development of methods for synthesizing compounds of Formula (I) as disclosed herein (e.g., oxyresveratrol), which produce oxyresveratrol compounds with high yields. The synthesized oxyresveratrol described herein also exhibits differing (e.g., higher) biological activity as compared with the commercially available biologically extracted oxyresveratrol.

In some aspects, the present disclosure provides a method for preparing a compound of Formula (I):

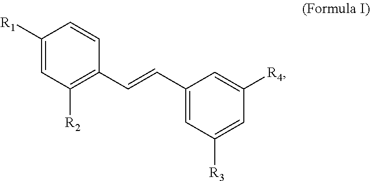

or a salt thereof. In Formula I, each of R₁, R₂, R₃ and R₄ independently can be H, —OH, —OMe, and —OEt; and at least one of R₁, R₂, R₃ and R₄, is —OH (unprotected). In specific examples, the Formula I compound can be

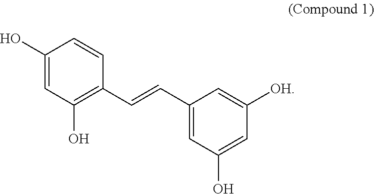 [SMILES: Oc1cc(O)cc(/C=C/c2ccc(O)cc2O)c1]

The method comprises: contacting a compound of Formula I-3:

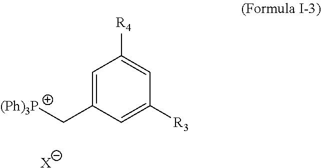

or a salt thereof with an aldehyde A1:

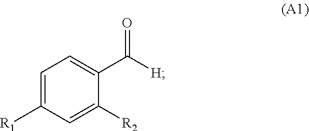

under conditions suitable to form a compound of Formula I or a salt thereof. In Formula I-3, X is a halide (e.g., Cl or Br, optionally Br).

In some embodiments, R₁ and R₂ are each —OH. Alternatively or in addition, In some R₃ and R₄ are each —OH.

In some embodiments, the molar ratio between the compound of Formula I-3 and the aldehyde A1 compound in the reaction system can range from 3:1 to 1:3. In some examples, the molar ratio between the compound of Formula I-3 and the aldehyde A1 compound in the reaction system can range from 2:1 to 1:2. In one example, the molar ratio between the two compounds can be about 1:1.5.

In some embodiments, the compound of Formula I-3 is:

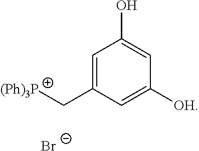

Alternatively, or in addition, the aldehyde A1 is:

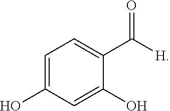

Exemplary suitable conditions for use in the contacting step for producing the Formula I compound with good yields include one or more of the following:
(a) Utilizing a suitable inorganic base (e.g., NaOC₆H₅, Na₂SiO₃, Ca(OH)₂, Mg(OH)₂, LiOH, Cs₂CO₃, K₃PO₄, t-buOK, K₂CO₃, and Na₂CO₃ (e.g., K₂CO₃, or Na₂CO₃) in the contacting step for producing the Formula I compound. In one example, the inorganic base can be Na₂CO₃. In some examples, the compound of Formula I-3 is contacted with the base prior to the contacting with aldehyde A1. In other examples, the compound of Formula I-3 is contacted with the base after the contacting with aldehyde A1. In yet other examples, the compound of Formula I-3 is contacted with the base concurrently with the contacting with aldehyde A1.
(b) Utilizing a suitable organic solvent such as n-methyl-2-pyrrolidone (NMP).
(c) Performing the reaction at a temperature of about 70-100 degrees Celsius, such as about 85-95 degrees Celsius. In some examples, a Formula I-3 compound and an aldehyde A1 can be heated to a temperature of about 85-95 degrees Celsius. In other examples, the Formula I-3 compound and the aldehyde A1 can be heated to a temperature of about 70-100 degrees Celsius (e.g., to a temperature of about 85-95° C.).

In specific examples, the contacting step for producing the compounds of Formula I is performed under all of the conditions (a)-(c).

In some embodiments, the compound of Formula I-3 used in the reaction for producing the Formula I compound disclosed herein may be prepared by process comprising: contacting a compound of Formula I-2:

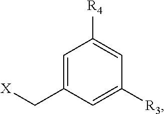

or a salt thereof with triphenylphosphine (PPh₃) at about 25-30 degrees Celsius under conditions suitable to form the compound of Formula I-3, or a salt thereof.

In some embodiments, the compound of Formula I-2 can be prepared by a process comprising: contacting a compound of Formula I-1:

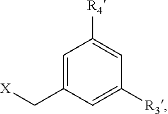

or a salt thereof, with boron tribromide (BBr₃) at about 25 degrees Celsius under conditions suitable to form a compound of the Formula I-2, or a salt thereof. In Formula I-1, each of R₃′ and R₄′ independently can be H, —OH, —OMe, or —OEt, and at least one of R₃′ and R₄′ is —OMe or —OEt, for example, —OMe.

In some embodiments, the compound of Formula I-1 is:

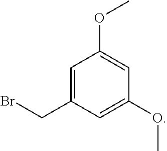

Also within the scope of the present disclosure is a compound of Formula I prepared by any of the synthesis methods disclosed herein.

The details of one or more embodiments of the invention are set forth in the description below. Other features or advantages of the present invention will be apparent from the following drawings and detailed description of several embodiments, and also from the appended claims.

### BRIEF DESCRIPTION OF THE DRAWINGS

The following drawings form part of the present specification and are included to further demonstrate certain aspects of the present disclosure, which can be better understood by reference to the drawing in combination with the detailed description of specific embodiments presented herein.

FIGS. 1A-1B shows particle size distributions (PSD) of oxyresveratrol evaluated using granulometry. FIG. 1A: commercially available oxyresveratrol (extracted from plants).

FIG. 1B: synthetic oxyresveratrol by the synthesis method disclosed in Example 1 below.

FIGS. 2A-2B show Differential Scanning Calorimetry (DSC) spectra of commercially available oxyresveratrol (FIG. 2A) and synthesized oxyresveratrol using methods described in Example 1 below (FIG. 2B).

FIGS. 3A-3B show Thermogravimetric Analysis (TGA) spectra of commercially available oxyresveratrol (FIG. 3A) and synthesized oxyresveratrol using methods described in Example 1 (FIG. 3B).

### DETAILED DESCRIPTION

### Definitions

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

As used herein, the term “treating” refers to the application or administration of a composition including one or more active agents to a subject, who has a target disease or disorder, a symptom of the disease/disorder, or a predisposition toward the disease/disorder, with the purpose to cure, heal, alleviate, relieve, alter, remedy, ameliorate, improve, or affect the disorder, the symptom of the disease, or the predisposition toward the disease or disorder.

Alleviating a target disease/disorder includes delaying the development or progression of the disease or reducing disease severity or prolonging survival. Alleviating the disease or prolonging survival does not necessarily require curative results. As used therein, “delaying” the development of a target disease or disorder means to defer, hinder, slow, retard, stabilize, and/or postpone progression of the disease. This delay can be of varying lengths of time, depending on the history of the disease and/or individuals being treated. A method that delays or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the disease. Development of the disease can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset. As used herein “onset” or “occurrence” of a target disease or disorder includes initial onset and/or recurrence.

As used herein, “an effective amount” refers to the amount of each active agent required to confer therapeutic effect on the subject, either alone or in combination with one or more other active agents. Determination of whether an amount of the antibody achieved the therapeutic effect would be evident to one of skill in the art. Effective amounts vary, as recognized by those skilled in the art, depending on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment.

The term “parenteral” as used herein includes subcutaneous, intracutaneous, intravenous, intramuscular, intraarticular, intraarterial, intrasynovial, intrasternal, intrathecal, intralesional, and intracranial injection or infusion techniques.

Compounds and methods of preparing compounds of this disclosure include those described generally above, and are illustrated by the classes, subclasses, and species disclosed herein. As used herein, the following definitions shall apply unless otherwise indicated. For purposes of this disclosure, the chemical elements are identified in accordance with the Periodic Table of the Elements, CAS version, Handbook of Chemistry and Physics, 75th Ed. Additionally, general principles of organic chemistry are described in “Organic Chemistry”, Thomas Sorrell, University Science Books, Sausalito: 1999, and “March's Advanced Organic Chemistry”, 5th Ed., Ed.: Smith, M. B. and March, J., John Wiley & Sons, New York: 2001, the entire contents of which are hereby incorporated by reference.

“Alkyl” refers to a linear, saturated, acyclic, monovalent hydrocarbon radical or branched, saturated, acyclic, monovalent hydrocarbon radical, having from one to three carbon atoms attached to the rest of the molecule by a single bond, e.g., methyl, ethyl, n-propyl, or 1-methylethyl (iso-propyl). An optionally substituted alkyl radical is an alkyl radical that is optionally substituted, valence permitting, by one, two, three, four, or five substituents independently selected from the group consisting of halo, cyano, nitro, oxo, hydroxyl, thio, or amino.

“Alkenyl” refers to a linear, acyclic, monovalent hydrocarbon radical or branched, acyclic, monovalent hydrocarbon radical, containing a carbon-carbon double bond, having two or three carbon atoms attached to the rest of the molecule by a single bond, e.g., ethenyl, or propenyl. An optionally substituted alkenyl radical is an alkenyl radical that is optionally substituted, valence permitting, by one, two, or three substituents independently selected from the group consisting of: halo, cyano, nitro, hydroxyl, thio, or amino.

“Alkynyl” refers to a linear, acyclic, monovalent hydrocarbon radical or branched, acyclic, monovalent hydrocarbon radical containing a triple bond and having two or three carbon atoms attached to the rest of the molecule by a single bond, e.g., ethynyl, or propynyl. An optionally substituted alkynyl radical is an alkynyl radical that is optionally substituted by one substituent selected from the group consisting of: halo, cyano, nitro, hydroxyl, thio, or amino.

“Alkoxy” refers to a radical of the formula —ORa where Ra is a hydrogen or an alkyl radical as defined above containing one to three carbon atoms. The alkyl part of the optionally substituted alkoxy radical is optionally substituted as defined above for an alkyl radical.

“Amino” refers to a radical of the formula —NRbRc where Rb and Rc are each hydrogen, or an alkyl radical as defined above containing one to three carbon atoms. The alkyl part of the optionally substituted amino radical is optionally substituted as defined above for an alkyl radical.

The term “Aryl” refers to to cyclic, aromatic hydrocarbon groups that have 1 to 3 aromatic rings, including monocyclic or bicyclic groups such as phenyl, biphenyl, or naphthyl. Where containing two aromatic rings (bicyclic, etc.), the aromatic rings of the aryl group may be joined at a single point (e.g., biphenyl), or fused (e.g., naphthyl). The aryl group may be optionally substituted by one or more substituents, e.g., 1 to 5 substituents, at any point of attachment. Exemplary substituents include, but are not limited to, —H, -halogen. —O—(C1-C6) alkyl, (C1-C6) alkyl, —O—(C2-C6) alkenyl, —O—(C2-C6) alkynyl, (C2-C6) alkenyl, (C2-C6) alkynyl, —OH, —OP(O)(OH)2, —OC(O)(C1-C6) alkyl, —C(O)(C1-C6) alkyl, and —OC(O)O(C1-C6) alkyl.

“Carbonyl” refers to a group of the Formula

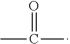

“Halogen” refers to a chloro, bromo, fluoro or iodo atom radical. The term “halogen” also contemplates terms “halo” or “halide.”

An “optionally substituted” moiety can be substituted with from one to four, or preferably from one to three, or more preferably one or two non-hydrogen substituents. Unless otherwise specified, when the substituent is on a carbon, it is selected from the group consisting of —OH, —CN, —NO₂, halogen, alkyl, heteroalkyl, cycloalkyl, heterocycle, aryl, heteroaryl, aralkyl, alkoxy, alkoxycarbonyl, alkanoyl, carbamoyl, substituted sulfonyl, sulfonate, sulfonamide and amino, none of which are further substituted. Unless otherwise specified, when the substituent is on a nitrogen, it is selected from the group consisting of alkyl, heteroalkyl, cycloalkyl, heterocycle, aryl, heteroaryl, aralkyl, alkoxy, alkoxycarbonyl, alkanoyl, carbamoyl, sulfonyl, sulfonate and sulfonamide none of which are further substituted.

Compounds of the present disclosure can exist as stereoisomers, wherein asymmetric or chiral centers are present. Stereoisomers are designated (R) or (S) depending on the configuration of substituents around the chiral carbon atom. The terms (R) and (S) used herein are configurations as defined in IUPAC 1974 Recommendations for Section E, Fundamental Stereochemistry, Pure Appl. Chem., (1976), 45: 13-30, hereby incorporated by reference. The present disclosure contemplates various stereoisomers and mixtures thereof and are specifically included within the scope of the present disclosure. Stereoisomers include enantiomers, diastereomers, and mixtures of enantiomers or diastereomers.

“Tautomer” is one of two or more structural isomers that exist in equilibrium and is readily converted from one isomeric form to another. This conversion results in the formal migration of a hydrogen atom accompanied by a switch of adjacent conjugated double bonds. Tautomers exist as a mixture of a tautomeric set in solution. In solutions where tautomerization is possible, a chemical equilibrium of the tautomers will be reached. The exact ratio of the tautomers depends on several factors, including temperature, solvent and pH. The concept of tautomers that are interconvertible by tautomerizations is called tautomerism.

As used herein, “reagent” is common partner of the reactant in many chemical reactions. It may be organic or inorganic; small or large; gas, liquid or solid. The portion of a reagent that ends up being incorporated in the product may range from all to very little or none.

“Thiol” refers to a radical of the formula —SRd where Rd is a hydrogen or an alkyl radical as defined above containing one to three carbon atoms. The alkyl part of the optionally substituted thiol radical is optionally substituted as defined above for an alkyl radical.

“Hydrophilic” as used herein refers to a molecule or molecules that have a hydrophilic-lipophilic balance of greater than 10, e.g., using the Griffin method.

### I. Synthetic Process

The present disclosure aims at developing improved synthesis methods for producing resveratrol compounds such as oxyresveratrol and derivatives thereof (e.g., compounds of Formula (I) disclosed herein) with improved yields compared with methods known in the literature. Conventional synthesis approaches for oxyresveratrol often involve protecting groups for —OH moieties in reacting agents. Such did not result in satisfactory yields of oxyresveratrol. Further, synthesis routes involving conventional Wittig reactions in the presence of bases such as MeONa, HMDSLi, LiOH, Cs₂CO₃, etc. also led to low production of oxyresveratrol.

The synthetic methods provided herein involve the use of reacting agents with unprotected —OH moieties and/or the use of inorganic bases such as Na₂CO₃. Surprisingly, the synthetic methods described herein led to successful production of the desired oxyresveratrol compound with high yields. The synthetic product also exhibited higher bioactivity as compared with commercially available oxyresveratrol (extracted from natural sources) as reported herein. Overall, the synthetic methods provided here show at least the following advantageous features: (a) high reaction efficiency, (b) high purity (e.g., 80% or higher) in the synthesized compounds; (c) low costs; and (d) no involvement of heavy metal catalysts such as palladium, thereby reducing or avoiding safety concerns for therapeutic uses.

### Resveratrol Compounds

A resveratrol compound provide herein may have the structure of Formula (I) or a salt thereof:

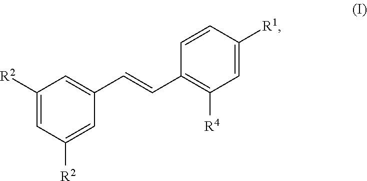

in which each of R¹, R², R³ and R⁴ is independently H, halo (e.g., F, Cl, or Br), hydroxyl, alkyl, alkenyl, alkynyl, alkoxy, thiol, or amine. In some embodiments, one or more of the R¹, R², R³ and R⁴ can be hydroxyl.

In some examples, a resveratrol compound may have the structure of Formula (Ia) or a salt thereof:

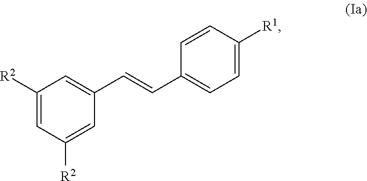

in which each of R¹, R², and R³ is as defined above.

In one example, the resveratrol compound is resveratrol (with all of R¹-R³ being —OH, and R⁴ being —H). In another example, the resveratrol compound is oxyresveratrol (with all of the R¹-R⁴ being —OH). A resveratrol compound as disclosed herein may have a suitable substitution at one or more suitable positions in resveratrol as known to those skilled in the art.

### Synthesis Methods

In some aspects, the disclosure provides a synthesis process for the preparation of a compound of Formula I or a salt thereof as described herein. An example of the synthesis scheme for a Formula (I) compound (e.g., oxyresveratrol), Scheme 1, is presented in Example 1 below.

In some embodiments, the synthesis method provided herein comprises a step (Step 3 exemplified in Scheme 1) of reacting a compound of Formula I-3

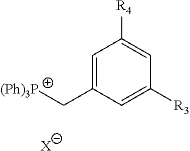

or a salt thereof with aldehyde A1 having the structure of Formula A1

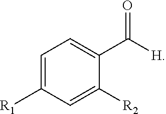

In these formulas, each of R₁, R₂, R₃ and R₄ is independently H, —OH, —OMe, or —OEt and at least one of R₁, R₂, R₃ and R₄ is —OH (i.e., unprotected). In some instances, at least two of R₁, R₂, R₃ and R₄ are the unprotected —OH moiety. In some instances, at least three of R₁, R₂, R₃ and R₄ are the unprotected —OH moiety. In one example, all of R₁, R₂, R₃ and R₄ are the unprotected —OH moiety. In Formula I-3, X is a halide. In some examples, X is Cl. In other examples, X is Br as exemplified in Scheme 1.

Step 3 described herein may be carried out under conditions allowing for formulation of a compound of Formula I or a salt thereof as provided herein. In some embodiments, a suitable molar ratio of the compound of Formula I-3 to the aldehyde A1 compound may be used to ensure successful production of the compound of Formula I with a desired yield. For example, the molar ratio of the compound of Formula I-3 to aldehyde A1 may range from 3:1 to 1:3, for example, 2:1 to 1:2. In one example, the molar ratio of the compound of Formula I-3 to aldehyde A1 is 2:1. In another example, the molar ratio of the compound of Formula I-3 to aldehyde A1 is 1.5:1. In yet other examples, the molar ratio of the compound of Formula I-3 to aldehyde A1 is 1:1. In some specific examples, the molar ratio of the compound of Formula I-3 to aldehyde A1 is 1:1.5. In other specific examples, the molar ratio of the compound of Formula I-3 to aldehyde A1 is 1:2.

Step 3 may be performed in the presence of an inorganic base. Suitable inorganic bases for use in any of the synthesis methods provided herein include LiOH, Cs₂CO₃, K₃PO₄, t-buOK, K₂CO₃, or Na₂CO₃. In one specific example, the inorganic base is Na₂CO₃. Use of Na₂CO₃ as the inorganic base in the synthesis method provided herein resulted in a high level of oxyresveratrol production and a low level of side products as reported in the Examples below.

In some instances, a compound of Formula I-3 as disclosed herein may be mixed with a suitable inorganic base as also disclosed herein, followed by addition of an aldehyde A1 in Step 3 to allow for production of the compound of Formula I. Alternatively, an aldehyde A1 as disclosed herein may be mixed with a suitable inorganic base as also disclosed herein, followed by addition of a compound of Formula I-3 in Step 3 to allow for production of the compound of Formula I. In one specific example, a compound of Formula I-3, an aldehyde A1, and a suitable inorganic base are mixed together concurrently in Step 3 to allow for production of the compound of Formula I.

Further, Step 3 may be carried out under a suitable temperature, for example, about 70-100 degree Celsius. In some examples, Step 3 is performed at a temperature of about 70-85 degree Celsius. In other examples, Step 3 is performed at a temperature of about 85-95 degree Celsius. In another examples, Step 3 is performed at a temperature of about 90-100 degree Celsius.

In one specific example, the compound of Formula I-3 is of the structure

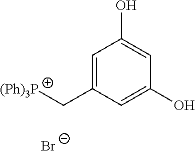

and the aldehyde A1 is of the structure

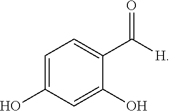

Step 3 is performed at a molar ratio of the compound of Formula I-3 to aldehyde A1 may be 1:1.5. Alternatively, or in addition, Step 3 is carried out in the presence of an inorganic base, which can be Na₂CO₃, at a temperature of about 85-95 degree Celsius.

Any suitable organic solvent may be used for dissolving the reagents used in the Step 3 reaction. Exemplary solvents include, but are not limited to, n-methyl-2-pyrrolidone (NMP), Acetonitrile, N,N-dimethylformamide (DMF), 1,4-dioxane, dimethoxyethane, tetrahydrofuran (THF, also known as oxolane), 2-methyloxolane (2-MeTHF). In one example, n-methyl-2-pyrrolidone (NMP) is used as an organic solvent in Step 3.

The reagents used in Step 3 described herein, including Formula I-3 compounds and aldehyde A1 compounds may be prepared by a conventional method or a process provided herein. In some embodiments, a Formula I-3 compound may be obtained by reaching a compound of Formula I-2

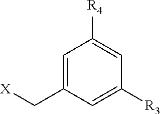

or a salt thereof with triphenylphosphine (PPh₃), for example Step 2 depicted in Scheme 1 below. In Formula I-2, X can be any halide provided herein. In one example, X is Br. R₃ and R₄ are as defined herein. In some instances, either R₃ or R₄, or both can be the unprotected —OH moiety. The molar ratio between the Formula I-2 compound to PPh₃ may range from 2:1 to 1:2. In one example, the molar ratio between the two reagents may be around 1:1 (e.g., 1:1.1). In some embodiments, a Formula I-3 compound may be obtained by reaching a compound of Formula I-2

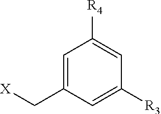

or a salt thereof with phosphite ester or tributyl phosphine (TBUP), for example Step 2 depicted in Scheme 1 below. In Formula I-2, X can be any of the halide provided herein. In one example, X is Br. R₃ and R₄ are as defined herein. In some instances, either R₃ or R₄, or both can be the unprotected —OH moiety. The molar ratio between the Formula I-2 compound to phosphite ester or tributyl phosphine (TBUP) may range from 5:1 to 1:5. In one example, the molar ratio between the two reagents may be around 1:2 (e.g., 1:2.5). In other example, the molar ratio between the two reagents may be around 1:3 (e.g., 1:2.9). The Step 2 reaction may be carried out under a suitable temperature, for example, about 20-35 degree Celsius. In some examples, Step 2 is carried under a temperature of about 25-30 degree Celsius. An organic solvent such as acetonitrile (ACN), toluene, or dichloromethane (DCM), may be used in the Step 2 reaction.

Any of the Formula I-2 compounds used as a substrate in Step 2 may be prepared via a conventional synthesis method or a method as disclosed herein. In some embodiments, a Formula I-2 compound may be prepared by reacting a compound of Formula I-1

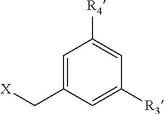

or a salt thereof with boron tribromide (BBr₃) to produce the Formula I-2 compound, for example Step 1 depicted in Scheme 1 below. R₃′ and R₄′ are each independently H, —OH, —OMe, or —OEt, and at least one of R₃′ and R₄′ is —OMe or —OEt. In some examples, both of R₃′ and R₄′ are —OMe. The molar ratio between the Formula I-1 compound to BBr₃ may range from 2:1 to 1:5. In some examples, the molar ratio between the Formula I-1 compound to BBr₃ may range from 1:1 to 1:4. In one example, the molar ratio between the two reagents may be around 1:3.

In some embodiments, a Formula I-2 compound may be prepared by reacting a compound of Formula I-1

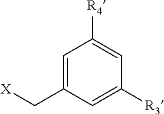

or a salt thereof with EtSNa, TMSI, MsOH, HBr or BCl₃ to produce the Formula I-2 compound, for example Step 1 depicted in Scheme 1 below. R₃′ and R₄′ are each independently H, —OH, —OMe, or —OEt, and at least one of R₃′ and R₄′ is —OMe or —OEt. In some examples, both of R₃′ and R₄′ are —OMe. The molar ratio between the Formula I-1 compound to EtSNa, TMSI, MsOH, HBr or BCl₃ may range from 5:1 to 1:10. In some examples, the molar ratio between the Formula I-1 compound to EtSNa, TMSI, MsOH, HBr or BCl₃ may range from 1:1 to 1:6. In one example, the molar ratio between the two reagents may be around 1:2. In other example, the molar ratio between the two reagents may be around 1:3. In other example, the molar ratio between the two reagents may be around 1:4. In yet another example, the molar ratio between the two reagents may be around 1:5. The Step 1 reaction may be carried out under a suitable temperature, for example, about 20-30 degrees Celsius. In some examples, Step 2 is carried under a temperature of about 25 degrees Celsius. An organic solvent such as dichloromethane (DCM), n-methyl-2-pyrrolidone (NMP), methanesulfonic acid, acetic acid, or a combination thereof, may be used in the Step 1 reaction.

In some embodiments, after each step of the reaction (e.g., Step 1, Step 2, and/or Step 3 described herein), the resulting compound can be isolated from the reaction mixture and purified (e.g., by column chromatography). Alternatively, after a preceding step (e.g., Step 1 and/or Step 2), the resultant intermediate compound can be used directly for the next step of reaction without isolation or purification.

Except as explicitly described, one or more steps of the synthesis method provided herein may include various bases and acids depending on the reaction performed. For example, Brönsted or Lewis bases or acids may also be used for the present process of preparation. Exemplary bases include, without limitation, triethylamine, pyridine, piperidine, 2,6-lutidine, pyrrolidine, toludine, diisopropylamine, diisopropyl ethylamine, sodium hydride, sodium hydroxide, and sodium carbonate. Exemplary Lewis acids according to the present invention include, without limitation, titanium tetrachloride, aluminum chloride, boron trifluoride, boron tribromide, dimethylboron bromide, phosphorous pentachloride, tin dichloride, and tin tetrachloride. In some embodiments, for example, the alkylation or alkenylation process is performed with one or more bases. Similarly, in some embodiments, certain reaction steps of the process of the disclosure may be carried out in the presence of a base (e.g., sodium hydride)

In further embodiments, nucleophilic or electrophilic additions and substitutions identified in the processes of preparation of the compounds of the present disclosure (including synthetic intermediate), various leaving or electrophilic groups have been used. Such groups include, without limitation, halogen, mesyl, triflate, acetyl, or tosyl.

In some embodiments, certain substrates used in each step of the reaction involved in the synthesis method may be prepared in a number of ways well known to those skilled in the art of organic synthesis. By way of example, such a substrate compound(s) used in the synthesis method of the present disclosure can be synthesized using the methods described herein, together with synthetic methods known in the art of synthetic organic chemistry, or variations thereon as appreciated by those skilled in the art. Standard synthetic methods and procedures for the preparation of organic molecules and functional group transformations and manipulations can be obtained from the relevant scientific literature or from standard textbooks in the field. Although not limited to any one or several sources, classic texts such as Smith, M. B., March, J., March's Advanced Organic Chemistry: Reactions, Mechanisms, and Structure, 5th edition, John Wiley & Sons: New York, 2001; Greene, T. W., Wuts, P. G. M., Protective Groups in Organic Synthesis, 3rd edition, John Wiley & Sons: New York, 1999; R. Larock, Comprehensive Organic Transformations, VCH Publishers (1989); L. Fieser and M. Fieser, Fieser and Fieser's Reagents for Organic Synthesis, John Wiley and Sons (1994); and L. Paquette, ed, Encyclopedia of Reagents for Organic Synthesis, John Wiley and Sons (1995), incorporated by reference herein, are useful and recognized reference textbooks of organic synthesis known to those in the art.

It is to be understood that the synthetic processes of the disclosure can tolerate a various functional groups, therefore various substituted starting materials can be used. The processes generally provide the desired final compound at or near the end of the overall process, although it may be desirable in certain instances to further convert the compound to a pharmaceutically acceptable salt thereof.

In the synthetic schemes described herein, compounds may be drawn with one particular configuration for simplicity. Such particular configurations are not to be construed as limiting the disclosure to one or another isomer, tautomer, regioisomer or stereoisomer, nor does it exclude mixtures of isomers, tautomers, regioisomers or stereoisomers, however, it will be understood that a given isomer, tautomer, regioisomer or stereoisomer may have a higher level of activity than another isomer, tautomer, regioisomer or stereoisomer.

Also with the scope of the present disclosure are Formula (I) compounds (e.g., oxyresveratrol) produced by the synthesis methods disclosed herein. Such synthetic Formula (I) compounds (e.g., synthetic oxyresveratrol) may be a mixture of the compound in anhydrate crystal form and the compound in hydrate crystal form. The synthetically produced compound was observed to have a lower fraction of the hydrate crystal form as compared with commercial oxyresveratrol derived from plant sources.

### II. Compositions Comprising Resveratrol-Type Compounds

In some embodiments are compositions comprising resveratrol-type compounds, e.g., produced by any of the synthesis methods disclosed herein. In some embodiments, the resveratrol-type compound is oxyresveratrol. In some embodiments, the resveratrol-type compound is a salt of oxyresveratrol. In some embodiments, the resveratrol-type compound is a hydrate of oxyresveratrol. In some embodiments, the resveratrol-type compound is a crystalline form of oxyresveratrol. In some embodiments, the composition comprises at least one crystalline form of oxyresveratrol.

In some embodiments, the compositions are cosmetic compositions. In some embodiments, the compositions are pharmaceutical compositions.

### Pharmaceutical Compositions

In some embodiments, the compositions are pharmaceutical compositions comprising a compound described herein, and a pharmaceutically acceptable excipient, diluent, or carrier.

In some embodiments, the compound described herein is an active agent. In some embodiments, any of the active agents, optionally the hydrophilic therapeutic agents, and the non-ionic surfactant, which may form micelles with the active agent and optionally the hydrophilic therapeutic agents, may be formulated into pharmaceutical compositions for use in the therapeutic applications disclosed herein.

In some embodiments, the pharmaceutical compositions disclosed herein comprise one or more of a resveratrol-type compound. In some embodiments, the pharmaceutical compositions described herein comprise a compound, wherein the compound is an active agent for alleviating conditions caused by abnormal subcutaneous deposit of adipose tissue or fat, e.g., lipoma or liposarcoma. See, e.g., WO2023/221945, the relevant disclosures of which are incorporated by reference for the subject matter and purpose referenced herein. The active agent as disclosed herein may form micelles with suitable non-ionic surfactants such as those disclosed herein (the first plurality of micelles). The pharmaceutical composition disclosed herein may further comprise a second plurality of micelles formed by a hydrophilic agent and a suitable non-ionic surfactant.

The pharmaceutical compositions to be used in the present methods can comprise pharmaceutically acceptable carriers, excipients, or stabilizers in the form of lyophilized formulations or aqueous solutions. (Remington: The Science and Practice of Pharmacy 20th Ed. (2000) Lippincott Williams and Wilkins, Ed. K. E. Hoover). Acceptable carriers, excipients, or stabilizers are nontoxic to recipients at the dosages and concentrations used, and may comprise buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™ PLURONICS™ or polyethylene glycol (PEG).

In other examples, the pharmaceutical composition described herein can be formulated in sustained-release format. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly (2-hydroxyethyl-methacrylate), or poly (vinyl alcohol)), polylactides (U.S. Pat. No. 3,773,919), copolymers of L-glutamic acid and 7 ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), sucrose acetate isobutyrate, and poly-D-(−)-3-hydroxybutyric acid.

The pharmaceutical compositions to be used for in vivo administration must be sterile. This is readily accomplished by, for example, filtration through sterile filtration membranes. Therapeutic antibody compositions are generally placed into a container having a sterile access port, for example, an intravenous solution bag or vial having a stopper pierceable by a hypodermic injection needle.

The pharmaceutical compositions described herein can be in unit dosage forms such as tablets, pills, capsules, powders, granules, solutions or suspensions, or suppositories, for oral, parenteral or rectal administration, or administration by inhalation or insufflation. For example, such pharmaceutical compositions may be formulated in a manner suitable for administration via a suitable route, for example, orally, parenterally, topically, intravenously, rectally, buccally, vaginally or via an implanted reservoir.

A sterile injectable composition, e.g., a sterile injectable aqueous or oleaginous suspension, can be formulated according to techniques known in the art using suitable dispersing or wetting agents (such as Polysorbate 80) and suspending agents. The sterile injectable preparation can also be a sterile injectable solution or suspension in a non-toxic parenterally acceptable diluent or solvent, for example, as a solution in 1,3-butanediol. Among the acceptable vehicles and solvents that can be employed are mannitol, water, Ringer's solution and isotonic sodium chloride solution. In addition, sterile, fixed oils are conventionally employed as a solvent or suspending medium (e.g., synthetic mono- or diglycerides). Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions can also contain a long-chain alcohol diluent or dispersant, or carboxymethyl cellulose or similar dispersing agents. Other commonly used surfactants such as Tweens or Spans or other similar emulsifying agents or bioavailability enhancers which are commonly used in the manufacture of pharmaceutically acceptable solid, liquid, or other dosage forms can also be used for the purposes of formulation.

In some embodiments, the compositions described herein may be formulated as a topical formulation, for example, a cream, lotion, or gel for topical application. Such cream, lotion, or gel may be formulated using ingredients known in the art to be appropriate for topical medications.

The carrier in the pharmaceutical composition must be “acceptable” in the sense of being compatible with the active ingredient of the formulation (and preferably, capable of stabilizing it) and not deleterious to the subject to be treated. For example, solubilizing agents such as cyclodextrins, which form more soluble complexes with the oxadiazole compounds, or more solubilizing agents, can be utilized as pharmaceutical carriers for delivery of the oxadiazole compounds. Examples of other carriers include colloidal silicon dioxide, magnesium stearate, sodium lauryl sulfate, and D&C Yellow #10.

In some embodiments, the pharmaceutical composition disclosed herein may further comprise an antioxidant. Examples include, but are not limited to, beta-carotene, lutein, lycopene, bilirubin, vitamin A, vitamin C (ascorbic acid), vitamin E, uric acid, nitric oxide, nitroxide, pyruvate, catalase, superoxide dismutase, glutathione peroxidases, N-acetyl cysteine, and naringenin, or a combination thereof.

### III. Therapeutic Uses of Formula I Compounds

In some aspects, provided herein are methods for alleviating (e.g., treating) conditions caused by abnormal subcutaneous deposit of adipose tissues and/or fat using any of the pharmaceutical compositions disclosed herein.

In some embodiments, conditions caused by abnormal subcutaneous deposit of adipose tissues and/or fat can be tumors of fat tissues, which can result in abnormal deposit of fat cells/tissues (adipose tissues) and/or fat under a skin spot (subcutaneous). In some examples, the tumor of fat tissues can be benign, for example, lipoma. In other examples, the tumor of fat tissues can be malignant, for example, liposarcoma. See, e.g., WO2023/221945, the relevant disclosures of which are incorporated by reference for the subject matter and purpose referenced herein.

For the purpose of the present disclosure, the appropriate dosage of the pharmaceutical composition as described herein will depend on the specific active agents and optionally hydrophilic agents employed, the type and severity of the disease/disorder, whether the composition is administered for preventive or therapeutic purposes, previous therapy, the patient's clinical history and response to the active agents, and the discretion of the attending physician. Typically, the clinician will administer a pharmaceutical composition disclosed herein, until a dosage is reached that achieves the desired result.

In some embodiments, the desired result is the reduction or elimination of a lipoma or liposarcoma, or alleviation of at least one symptom associated with a lipoma-associated disease (e.g., pain) or a liposarcoma-associated disease. Methods of determining whether a dosage resulted in the desired result would be evident to one of skill in the art. The particular dosage regimen, i.e., dose, timing and repetition, used in the method described herein will depend on the particular subject and that subject's medical history. Treatment efficacy for a target disease/disorder can be assessed by methods well-known in the art.

Conventional methods, known to those of ordinary skill in the art of medicine, can be used to administer the pharmaceutical composition to the subject, depending upon the type of disease to be treated or the site of the disease. This composition can also be administered via other conventional routes, e.g., administered parenterally, by topically or via an implanted reservoir. In addition, it can be administered to the subject via injectable depot routes of administration such as using 1-, 3-, or 6-month depot injectable or biodegradable materials and methods.

In one embodiment, a pharmaceutical composition disclosed herein is administered via site-specific or targeted local delivery techniques. Examples of site-specific or targeted local delivery techniques include various implantable depot sources of the composition or local delivery catheters, such as infusion catheters, an indwelling catheter, or a needle catheter, synthetic grafts, adventitial wraps, shunts and stents or other implantable devices, site specific carriers, direct injection, or direct application.

In some examples, the pharmaceutical composition disclosed herein is administered to a subject by injection, for example, intramuscular (IM) injection, subcutaneous (SC) injection, intravenous (IV) injection, intraosseous injection, epidural injection, intradermal (ID) injection, or any other injected forms.

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

The practice of the present disclosure will employ, unless otherwise indicated, conventional techniques of molecular biology (including recombinant techniques), microbiology, cell biology, biochemistry, chemistry, and immunology.

### Example 1: Synthesis of Oxyresveratrol

Introducing protecting groups to hydroxy groups in reacting substrates exhibited challenges in stages of Wittig reaction and deprotection. As such, this Example explores the use of unprotected substrates in synthesizing oxyresveratrol via Wittig reaction. Protecting groups such as methyl groups on hydroxy groups in reaction substrates were removed in advance. The synthesis scheme is exemplified in Scheme 1 below.

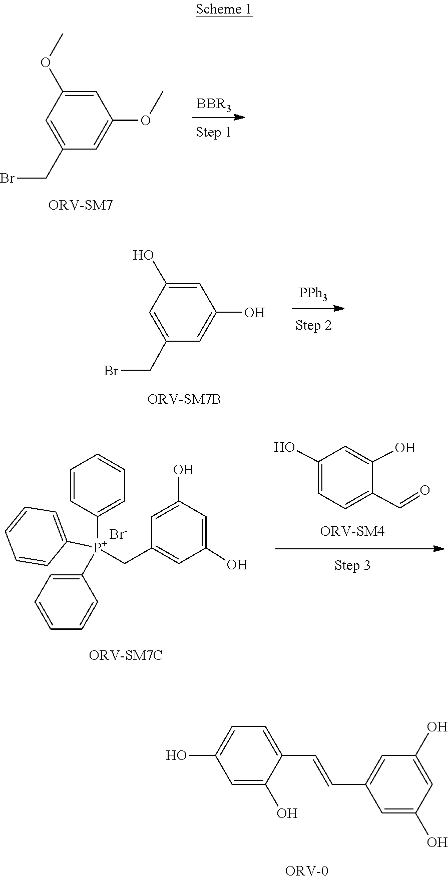

### Step 1: Preparation of ORV-SM7B

ORV-SM7 (1.0 eq.), BBr3 (3.0 eq.) and DCM (10.0 v) were reacted at 25° C. Dichloromethane (5L, 5.0 v) and ORV-SM7 (1.0 Kg, 1.0 eq.) were then added to the reaction mixture under nitrogen protection. The reaction was then cooled to 0±5° C. and boron tribromide (2.3 Kg, 2.1 eq.) was added dropwise. After the dropwise addition, the temperature was raised to 25±5° C. for about 2 hours. To the reaction mixture purified water (5.0 L, 5.0 v) was added to another reaction flask and the mixture was cooled to 0±5° C. The reaction mixture was then dripped into ice water where white solids gradually precipitated. The reaction efficiency was analyzed by HPLC sampling. For comparison, ORV-SM7 was used as a standard, the peak area of ORV-SM7 is ≤1%).

ORV-SM7B was separated from the reaction mixture without further purification by adding ORV-SM7B into ice water, stirring the mixture, and filtering. The precipitate comprised ORV-SM7B.

### Step 2: Preparation of ORV-SM7C

ORV-SM7B (1.0 eq.), PPh₃ (1.1 eq.), and ACN (10.0 v) were reacted at a temperature of 25-30° C. ACN (10 L, 10.0 v) and ORV-SM7B (wet filter cake, 1.0 eq.) were added to the reaction flask in sequence under nitrogen protection. Keep at 25±5° C., stir for 10-20 minutes, filter, and rinse the filter cake twice with ACN (2 L, 2.0 v). Collect the filtrate and add PPh₃ (1.25 Kg, 1.1 eq.). Adjust temperature to 25±5° C. and stir for at least 2 hours. Filter, and the filter cake is rinsed twice with ACN (1 L, 1.0 v). Collect the filter cake and dry it under vacuum at 25±5° C. until LOD≤2%. The collected off-white solid ORV-SM7C.

For HPLC sampling, the standard is that the peak area of ORV-SM7B is ≤1%.

ORV-SM7C was separated and purified as follows. The reaction system was filtered directly and the filter cake contains ORV-SM7C. ORV-SM7C was further purified via NMP and MTBE crystallization. The NMR characterization of ORV-SM7C is as follows: ¹H NMR (300 MHz, Methanol-d₄) δ 7.95-7.82 (m, 3H), 7.79-7.64 (m, 9H), 7.69-7.59 (m, 4H), 6.21 (q, J=2.2 Hz, 1H), 5.94 (t, J=2.3 Hz, 2H), 4.72 (d, J=14.8 Hz, 2H).

### Step 3: Preparation of ORV

The Step 3 reaction is detailed below:
1. Add NMP (12 L, 10.0 v), ORV-SM4 (534 g, 1.5 eq.) and ORV-SM7C (1.2 Kg, 1.0 eq.) into the reaction kettle in sequence under nitrogen protection.
2. Stir for 5-10 minutes to dissolve and clarify the system, then add Na₂CO₃ (1.37 Kg, 5.0 eq.).
3. Heat the reaction system to 100±5° C. and protect from light for at least 16 hours.
4. For sampling and testing, the standard is that the peak area of ORV-SM7C is ≤5%.
5. Prepare 20% acetic acid solution (12 L, 10.0 v) and 36% sodium chloride aqueous solution (12 L, 10.0 v).
6. Add the above two prepared solutions to 2-MeTHF (12 L, 10.0 v), and cool down to 5±5° C.
7. Control the temperature at 5±5° C., drop the reaction system into the three mixed solutions to quench, separate the liquids, and collect the organic phase.
8. The aqueous phase was extracted with 2-MeTHF (9.6 L*2, 8.0 v*2).
9. Sampling water phase analysis and testing, the standard is ORV-0 content ≤2%, if not qualified, continue extraction with 2-MeTHF (9.6 L, 8.0 v) until qualified.
10. Combine all organic phases and wash with 20% sodium chloride solution (12 L*2, 10.0 v*2), and collect the organic phases by liquid separation.
11. Concentrate the organic phase to 2-3 volumes and take a sample for H-NMR testing to calculate the volume of NMP relative to ORV-0.
12. Add NMP to 4 volumes and slowly drop in MTBE (24 L, 20.0 v).
13. Cool the system to 0±5° C. and continue stirring for at least 2 hours to crystallize.
14. Filter, rinse the MTBE (2.4 L*2, 2.0 v*2) filter cake, and take a sample to detect the peak area of standard TPPO by HPLC≤0.5%.
15. If it is unqualified, add the filter cake to NMP (2.4 L, 2.0 v), raise the temperature to 45-50° C., dissolve and clarify, add MTBE (12 L, 10.0 v) dropwise for crystallization until it is qualified.
16. The qualified filter cake was dissolved in 2-MeTHF (6 L, 5.0 v) and washed with 5% sodium chloride aqueous solution (6 L*2, 5.0 v*2).
17. The washed organic phase was collected and concentrated to 10 volumes with EtOH (6 L*3, 5.0 v*3).
18. Add activated carbon (0.36 Kg, 0.3 wt.) to decolorize for at least 2 hours and filter.
19. The filtrate is concentrated to 2-3 volumes.
20. Control the temperature to 35±5° C. and slowly drop H₂O (16.8 Kg, 14.0 v). After the dropwise addition, cool down to 0±5° C.
21. Filter and rinse the filter cake with H₂O (1.2 Kg*2, 1.0 v*2).
22. Collect the filter cake, heat it up to 50±5° C., and vacuum dry the material to LOD=2.0%.

The reaction efficiency was analyzed by HPLC sampling. The standards are: ORV-0≥98.0%; single impurity≤0.5%.

ORV-0 Purification conditions:

First purification: Add the crude product to NMP (2.0 v), raise the temperature to 45-50° C. to dissolve and clarify, add MTBE (10.0 v) dropwise, and cool to 0±5° C. for crystallization.

Second purification: Dissolve the crude product in ethanol (2.0 v), raise the temperature to 35±5° C. and slowly drip in H₂O (14.0 v). After the dropwise addition, cool down to 0±5° C. for crystallization.)

ORV-0 NMR Characterization: ¹H NMR (300 MHz, DMSO-d₆) δ 9.59 (s, 1H), 9.41 (s, 1H), 9.16 (s, 2H), 7.35 (d, J=8.5 Hz, 1H), 7.16 (d, J=16.5 Hz, 1H), 6.77 (d, J=16.5 Hz, 1H), 6.34 (dd, J=6.0, 2.2 Hz, 3H), 6.25 (dd, J=8.4, 2.4 Hz, 1H), 6.08 (t, J=2.1 Hz, 1H).

Table 1 summarizes the synthesis efficiency.

TABLE 1
Reaction Trials and % Yields

| | Starting | | Auxiliary | | Temp | Results (%) | |
|---|---|---|---|---|---|---|---|
| Entry | Material | Solvent | Material | Base | (° C.) | ORV-0A* | ORV-0 |
| 1 | ORV- | NMP (10 v) | ORV-SM4 | DBU | 85-95 | N.A. | |
| | SM7C | | (1.5 eq.) | (2.0 eq.) | | | |
| 2 | ORV- | NMP (10 v) | ORV-SM4 | DMAP | 85-95 | No Reaction | |
| | SM7C | | (1.5 eq.) | (2.0 eq.) | | | |
| 3 | ORV- | NMP (10 v) | ORV-SM4 | TEA.LiCl | 85-95 | No Reaction | |
| | SM7C | | (1.5 eq.) | (2.0 eq.) | | | |
| 4 | ORV- | NMP (10 v) | ORV-SM4 | Li₂CO₃ | 85-95 | No Reaction | |
| | SM7C | | (1.5 eq.) | (2.0 eq.) | | | |
| 5 | ORV- | NMP (10 v) | ORV-SM4 | Lutidine. LiCl | 85-95 | No Reaction | |
| | SM7C | | (1.5 eq.) | (2.0 eq.) | | | |
| 6 | ORV- | NMP (10 v) | ORV-SM4 | MeONa | 85-95 | Majority | n.a |
| | SM7C | | (1.5 eq.) | (2.0 eq.) | | | |
| 7 | ORV- | NMP (10 v) | ORV-SM4 | HMDSLi | 85-95 | Majority | n.a |
| | SM7C | | (1.5 eq.) | (2.0 eq.) | | | |
| 8 | ORV- | NMP (10 v) | ORV-SM4 | LiOH | 85-95 | Majority | n.a |
| | SM7C | | (1.5 eq.) | (2.0 eq.) | | | |
| 9 | ORV- | NMP (10 v) | ORV-SM4 | Cs₂CO₃ | 85-95 | Majority | n.a |
| | SM7C | | (1.5 eq.) | (2.0 eq.) | | | |
| 10 | ORV- | NMP (10 v) | ORV-SM4 | K₃PO₄ | 85-95 | 43.4 | 4.2 |
| | SM7C | | (1.5 eq.) | (2.0 eq.) | | | |
| 11 | ORV- | NMP (10 v) | ORV-SM4 | t-BuOK | 85-95 | 46.8 | 10.2 |
| | SM7C | | (1.5 eq.) | (2.0 eq.) | | | |
| 12 | ORV- | NMP (10 v) | ORV-SM4 | K₂CO₃ | 85-95 | 8.6 | 73.3 |
| | SM7C | | (1.5 eq.) | (2.0 eq.) | | | |
| 13 | ORV- | NMP (10 v) | ORV-SM4 | Na₂CO₃ | 85-95 | 4.5 | 80.0 |
| | SM7C | | (1.5 eq.) | (2.0 eq.) | | | |

*ORV-0A (structure shown below) refers to byproduct 5-methylbenzene-1,3-diol.
ORV-0A

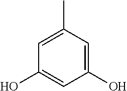

### Example 2: Solid Forms Analysis of Oxyresveratrol

Samples of biologically extracted oxyresveratrol were characterized and analyzed in comparison with oxyresveratrol samples synthesized using the methods described herein. The oxyresveratrol samples comprise both crystal form I and crystal form III, but the proportions are different. Crystal form I is an anhydrous crystal, and crystal form III is a hydrate crystal. The commercially available extraction source raw materials have a higher proportion of hydrate crystal, and the water of crystallization signal in the raw materials can be seen in the DSC spectrum.

### XRPD Data

X-ray powder diffraction (XRPD) measurements were carried out to evaluate biologically extracted oxyresveratrol (C) in comparison to synthesized oxyresveratrol (D) using methods described herein. Measurements were carried out after dynamic vapor sorption (DVS) and drying after 150° C. Table 2 below lists peaks (2-θ) of Sample A (Form III), Sample B (Form I), Sample C (commercially purchased), and Sample D (synthesized by the above-described process).

TABLE 2
Characteristic XRPD Peaks

| Samples | XRPD Peaks (2-θ) |
|---|---|
| A | 27.548°, 20.271°, 14.915°, 23.994°, 17.821°, 21.272°, 29.745°, |
| | 10.052°, 25.218°, 14.476°, 30.418°, 24.843°, 18.913°, and |
| | 23.113° |
| B | 21.445°, 14.557°, 22.378°, 26.663°, 27.158°, 19.592°, 19.268°, |
| | 20.210°, 21.919°, 28.495°, 13.466°, 17.675°, 22.906°, 25.719°, |
| | and 28.035° |
| C | 21.451°, 14.56°, 22.379°, 26.663°, 27.613°, 27.177°, 19.583°, |
| | 20.251°, 19.274°, 21.922°, 28.52°, 13.446°, 17.66°, 22.916°, |
| | 25.728°, and 28.022° |
| D | 21.409°, 14.525°, 22.345°, 27.611°, 26.625°, 27.136°, 19.229°, |
| | 20.229°, 19.559°, 28.487°, 17.634°, 13.42°, 21.885°, 22.874°, |
| | 25.671°, and 7.206° |

### PSD Data

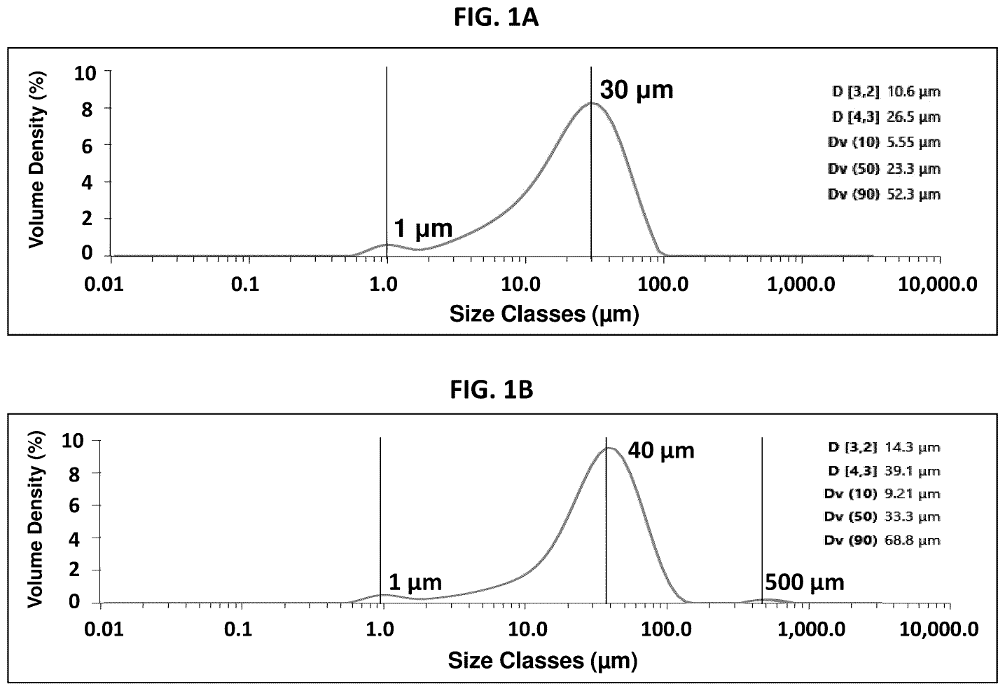

Particle size distributions (PSD) of synthesized oxyresveratrol using methods described herein (bottom) and biologically extracted oxyresveratrol (top) were evaluated using granulometry as shown in FIGS. 1A and 1B.

### DSC Data

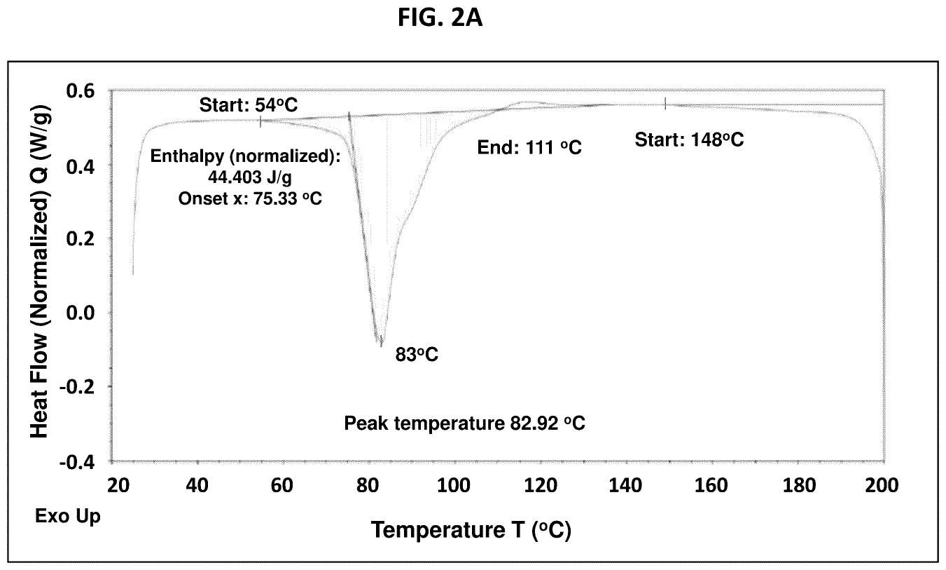

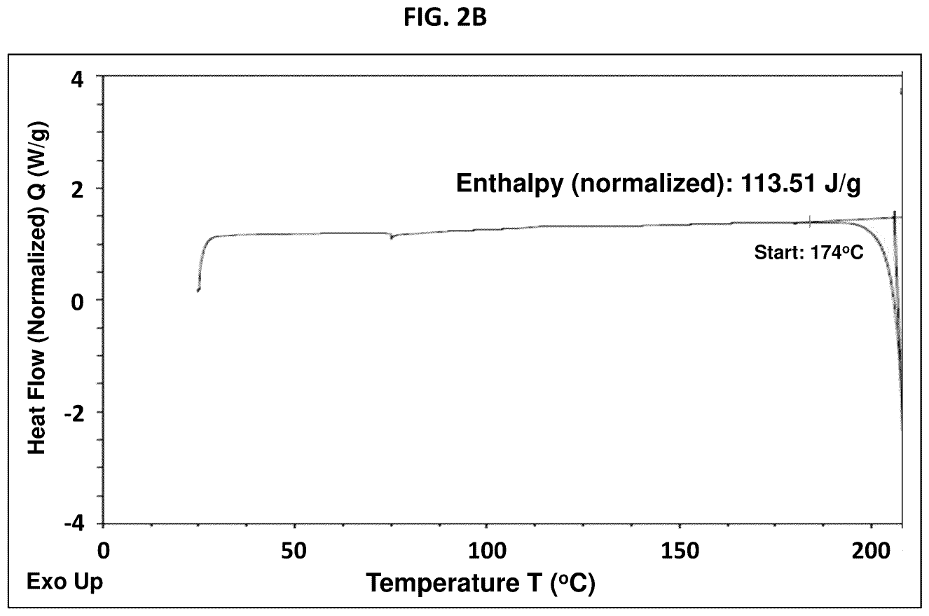

Differential Scanning Calorimetry (DSC) was also carried out on C and D to determine crystalline content. As shown in FIG. 2A demonstrated a normalized enthalpy of 44.403 J/g at a peak temperature of 82.92 degrees Celsius and FIG. 2B demonstrated a normalized enthalpy of 113.51 J/g at over 200 degrees Celsius. The water of crystallization signal in C sample is observed at about 83 degrees in the DSC spectrum. The downward peak near 83° is a characteristic signal showing presence of the hydrate crystal form.

### TGA Data

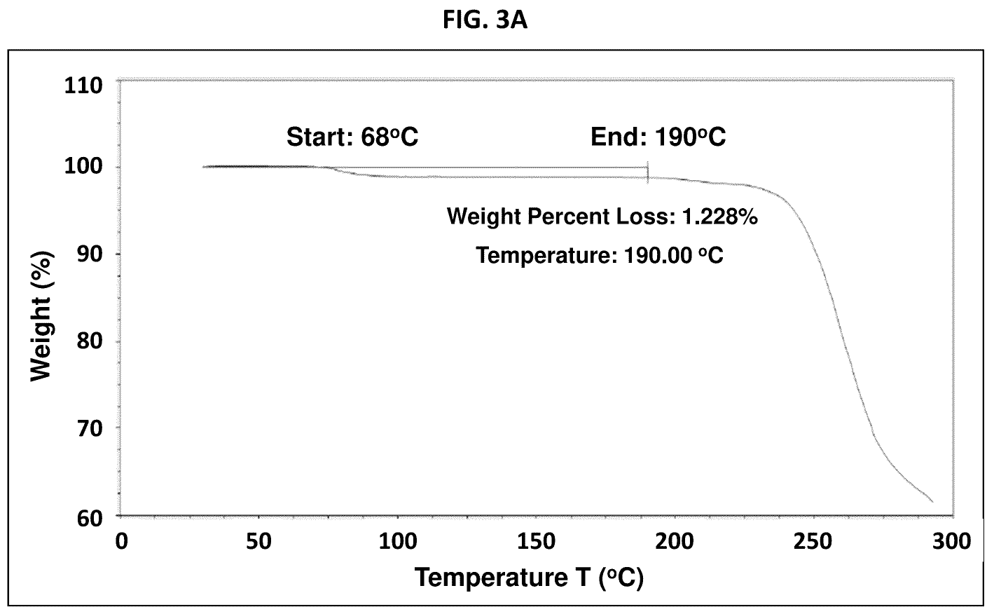

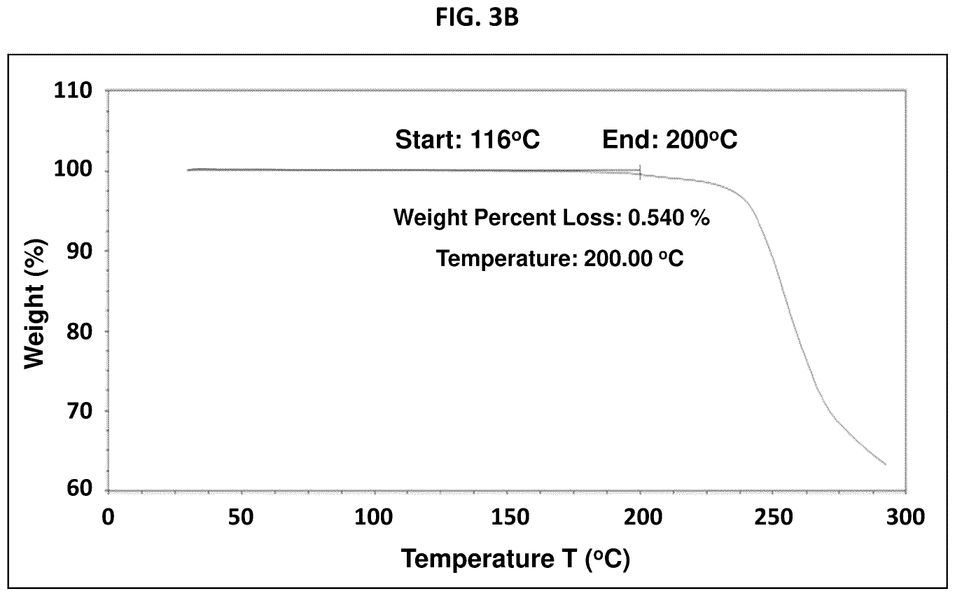

Thermogravimetric Analysis (TGA) was carried out for C and D as shown in FIGS. 3A and 3B. C demonstrated a weight percent loss of 1.228% at 190.00 degrees Celsius and D demonstrated a weight percent loss of 0.540% at 200 degrees Celsius.

### Example 3: Bioactivity in Inhibiting Melanin Production

This example explores the bioactivity of the synthesized oxyresveratrol compound in inhibiting melanin production as relative to commercial oxyresveratrol extracted from plant sources. The test conditions are summarized in Table 3 below.

TABLE 3
Testing Groups

| | | | | α-MSH | Conc. | |
|---|---|---|---|---|---|---|
| No. | Group | N | Vehicle | (0.1 μM) | (μM) | Treatment Time |
| 1 | Vehicle | 3 | 0.2% DMSO | − | − | α-MSH treat 24 |
| 2 | α-MSH | 3 | | + | − | hours + |
| 3 | Commercial-32 | 3 | | + | 32 | TA treat 24 |
| 4 | Commercial -128 | 3 | | + | 128 | hours |
| 5 | Synthetic-32 | 3 | | + | 32 | |
| 6 | Synthetic-128 | 3 | | + | 128 | |

As shown in Tables 4 and 5 below, oxyresveratrol, both commercial and synthetic, inhibited melanin production induced by α-MSH. The synthetic resveratrol showed greater inhibitory activity, e.g., at 128 μM, as compared with the commercial resveratrol.

TABLE 4
Inhibition of Melanin by Oxyresveratrol at 32 μM
Melanin Content (%)

| | | | Commercial | Synthetic |
|---|---|---|---|---|
| Group | Vehicle | α-MSH | 32 μM | 32 μM |
| TA conc. (μM) | 0.2% DMSO | 0.2% DMSO | 32 | 32 |
| α-MSH | − | + | + | + |
| Mean | 54.6 | 100.0 | 77.4 | 75.7 |
| SD | 3.4 | 1.7 | 3.7 | 3.0 |
| t-test | 0.0000 | 1.0000 | 0.0006 | 0.0003 |
| t-test vs 32 μM | | | 1.0000 | 0.5753 |

TABLE 5
Inhibition of Melanin by Oxyresveratrol at 128 μM
Melanin content (%)

| | | | Commercial | Synthetic |
|---|---|---|---|---|
| Group | Vehicle | α-MSH | 128 μM | 128 μM |
| TA conc. (μM) | 0.2% DMSO | 0.2% DMSO | 128 | 128 |
| α-MSH | − | + | + | + |
| Mean | 54.6 | 100.0 | 45.5 | 38.5 |
| SD | 3.4 | 1.7 | 4.0 | 3.3 |
| t-test | 0.0000 | 1.0000 | 0.0000 | 0.0000 |
| t-test vs 128 μM | | | 1.0000 | 0.0775 |

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

What is claimed is:

1. A method of preparing a compound of Formula I: 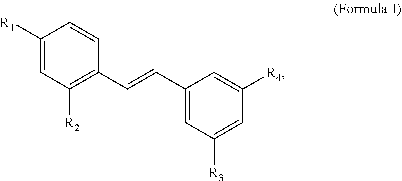
or a salt thereof, the method comprising: contacting a compound of Formula I-3: 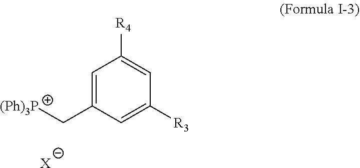
or a salt thereof with aldehyde A1: 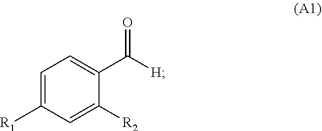
under conditions suitable to form a compound of Formula I or a salt thereof; and
wherein R₁, R₂, R₃ and R₄ are each independently selected from the group consisting of: H, —OH, —OMe, and —OEt;
wherein at least one of R₁, R₂, R₃ and R₄, is —OH; and
wherein X is a halide.

2. The method according to claim 1, wherein R₁ and R₂ are each —OH.

3. The method according to claim 1, wherein R₃ and R₄ are each —OH.

4. The method according to claim 1, wherein the compound of Formula I-3 and aldehyde A1 have a molar ratio of about 3:1 to 1:3, optionally about 1:1.5.

5. The method according to claim 1, wherein the Formula I-3 compound is:

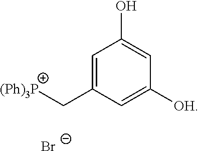

6. The method according to claim 1, wherein the aldehyde A1 is:

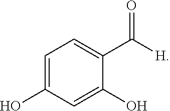

7. The method according to claim 1, wherein the compound of Formula I is:

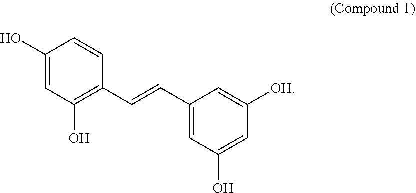 [SMILES: Oc1cc(O)cc(/C=C/c2ccc(O)cc2O)c1]

8. The method according to claim 1, wherein the contacting step is performed in the presence of an inorganic base.

9. The method of claim 8, wherein the inorganic base is selected from the group consisting of: NaOC₆H₅, Na₂SiO₃, Ca(OH)₂, Mg(OH)₂, LiOH, Cs₂CO₃, K₃PO₄, t-buOK, K₂CO₃, and Na₂CO₃.

10. The method of claim 8, wherein the inorganic base is Na₂CO₃ or K₂CO₃, optionally Na₂CO₃.

11. The method according to claim 8, wherein the compound of Formula I-3 is contacted with the base prior to the contacting with the aldehyde A1.

12. The method according to claim 8, wherein the compound of Formula I-3 is contacted with the base after the contacting with the aldehyde A1.

13. The method according to claim 8, wherein the compound of Formula I-3 is contacted with the base concurrently with the contacting with the aldehyde A1.

14. The method according to claim 1, wherein the contacting step is performed in the presence of an organic solvent.

15. The method according to claim 14, wherein the organic solvent is n-methyl-2-pyrrolidone (NMP).

16. The method according to claim 1, wherein the contacting step is performed at a temperature of about 70-100 degrees Celsius.

17. The method according to claim 16, wherein the contacting step is performed at a temperature of about 85-95 degrees Celsius.

18. The method according to claim 1, wherein the compound of Formula I-3 is prepared by a method comprising:
contacting a compound of Formula I-2: 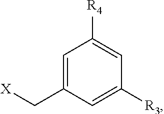
or a salt thereof, with triphenylphosphine (PPh₃) at about 25-30 degrees Celsius under conditions suitable to form the compound of Formula I-3, or a salt thereof.

19. The method according to claim 18, wherein, the compound of Formula I-2 is prepared by a method comprising:
contacting a compound of Formula I-1: 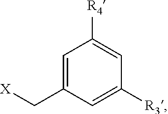
or a salt thereof, with BBr₃ at about 25 degrees Celsius under conditions suitable to form a compound of Formula I-2, or a salt thereof, wherein R₃′ and R₄′ are each independently H, —OH, —OMe, or —OEt, and wherein at least one of R₃′ and R₄′ is —OMe or —OEt.

20. The method of claim 19, wherein the compound of Formula I-1 is:

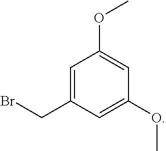

21. A compound of Formula (I), 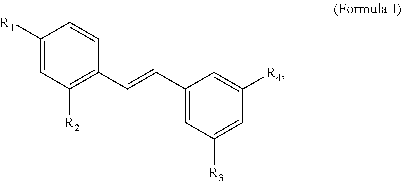
or a salt thereof, which is produced by the method according to claim 1;
wherein R₁, R₂, R₃ and R₄ are each independently selected from the group consisting of: H, —OH, —OMe, and —OEt; and
wherein at least one of R₁, R₂, R₃ and R₄, is —OH.
